(19) United States
(12) Patent Application Publication
(10) Pub. No.: US 2003/0091513 A1
(43) Pub. Date: May 15, 2003

(54) Method to generate water soluble or nonwater soluble in nanoparticulates directly in suspension or dispersion media

(76) Inventors: Nahed M. Mohsen, Plymouth, MI (US); Thomas A. Armer, Cupertino, CA (US)

Correspondence Address: FITZPATRICK CELLA HARPER & SCINTO, 30 ROCKEFELLER PLAZA, NEW YORK, NY, 10112, US

(21) Appl. No.: 10/264,030

(22) Filed: Oct. 3, 2002

Related U.S. Application Data
(60) Provisional application No. 60/326,442, filed on Oct. 3, 2001.

(51) Int. Cl. A61K 38/28; A61L 9/04; A61K 9/14; A61K 31/48; A61K 31/573

(52) U.S. Cl. 424/46; 514/3; 514/179; 514/288

(57) ABSTRACT

A method for preparing a formulation containing nanoparticles of a compound is described. The method includes forming the compound into nanoparticles and then delivering the nanoparticles directly to a collection media. The collection media is a desired component of the formulation.

### CONTINUITY DATA

This application claims benefit and incorporates by reference the entire disclosure of provisional application No. 60/326,442, filed Oct. 3, 2001.

### BACKGROUND OF THE INVENTION

Use of nanoparticles of various compounds, including medicaments and pigments, is well-known for making useful suspensions of non-water soluble materials. Examples of such useful suspensions include pharmaceutical formulations and paints.

Conventionally, nanoparticles to be included in foods, cosmetics, pharmaceutical formulations, inks and paints are generated using a variety of known techniques and collected for later combination with suitable carriers, suspending agents and the like.

However, there is a tendency for nanoparticles to agglomerate. Thus, when it becomes necessary to later suspend the particles, one must overcome the forces of agglomeration, which takes additional time and energy. Further, it is possible that the particles no longer have the same size and morphology they had upon their formation.

Accordingly, methods to improve the preparation of nanoparticles into useful formulations are needed.

### DETAILED DESCRIPTION OF THE INVENTION

The present invention provides a method to generate nanoparticulates directly in the dispersing or suspending liquid fluid carrier. The invention teaches that nanoparticles can be made using known particle generation methods, including precipitation, volume-exclusion precipitation, spray drying and Super-critical Fluid (SCF), using any SCF process including RESS, SEDS, etc. It specifically excludes grinding, milling or similar means of mechanical attrition at taught in U.S. Pat. No. 6,264,922. In accordance with this invention, the resulting nanoparticles are collected directly into any condensed fluid or any collection media that is also a component of the final desired suspensed or dispersed formulation. Such a process directly generates nanoparticles (those having hydrodynamic radii less than 1.0 micron) into suspensions or dispersions that can be used to formulate useful compositions such as inks, paints , foods, cosmetics or pharmaceutical compositions. Such suspensions or dispersions are used to produce different formulations.

More specifically, the invention provides a process that generates nanoparticulates from numerous water soluble or non-water soluble compounds that can be directly fabricated into dispersions or suspensions. Dispersions are defined as two phase solid-liquid mixtures where the liquid is the disperion media and the solid is the dispersed media. The solid phase particles being having hydrodynamic (or settling) radii generally less than 0.500 microns (or 500 nanometers). Typical dispersion media be water, alcoholic aqueous solutions, organic liquids, condensed gases such as fluorocarbon propellants, carbon dioxide or alkanes. Typical dispersed media would be drug compounds, ink or paint pigments, food compounds or cosmetics.

Suspensions are defined as two phase solid-liquid mixtures where the liquid is the suspension media and the solid is the suspended media. The solid phase particles being having hydrodynamic (or settling) radii generally greater than 500 nanometers. Typical suspension media be water, alcoholic aqueous solutions, organic liquids, condensed gases such as fluorocarbon propellants, carbon dioxide or alkanes. Typical suspended media would be drug compounds, ink or paint pigments, food compounds or cosmetics.

The present invention also provides a method to obtain dispersions or suspensions that are subsequently utilized to formulate oral, pulmonary, parental and diagnostic pharmaceutical formulations. These pharmaceutical formulations include nanoparticulate medicaments (nanomedicaments) which can be selected from among anti-allergic, anti-inflammatory, steroid, anti-cholinergic, mucolytic, and/or beta-agonist agents, or combinations thereof.

As further examples of suitable pharmaceutical formulations, the suspended or dispersed phase nanomedicaments can be selected from the group consisting of salbutamol, salmeterol, formeterol, fenterol, fluticasone dipropionate, beclomethasone dipropionate, dexamethasone, budesonide, ciclesonide, flunisolide, triamcinolone, sodium cromolyn, ipratropium and their salts or solvates. The suitable pharmaceutical agent may also be any two or more combinations of these exemplified medicaments.

Other examples of nanomedicaments which can be added to a useful pharmaceutical formulations are anti-cancer, anti-emetic, anti-migraine, narcotic analgesic, antipsychotic, anti-depressant, analgesic, anti-inflammatory, antineoplastic, antibiotic, anti-infective, or antidiuretic agents.

Also encompassed within the scope of the present invention are dispersion or suspension formulations wherein the nanomedicament is a protein and/or a peptide which can be used to treat respiratory or systemic disorders or diseases.

This invention provides specifically for a process that generates nanoparticulates for water-soluble agents. For example, the compound dihydroergotamine, or the compound formoterol can be condensed by RESS methods directly into HFA or CFC propellants to form stable particulate suspensions and dispersions.

A method to aerosolize nanoparticulate dispersions or suspensions that are fabricated in accordance with the process described herein, which use nonaqueous propellant based delivery systems, dry powder delivery systems or aqueous media based delivery systems are also within the scope of this invention.

By use of the method, nanoparticulates with optimum particle design in an optimum delivery system can be obtained to achieve efficient drug delivery to the respiratory tract, including the mouth, nose, throat, upper airways, deep lung, and systemic circulation via the deep lung, in order to treat local disorders and diseases. Particles must be in a size range of: less than 20 microns to reach any part of the respiratory tract in appreciable quantities; less than 10 microns to reach beyond the naso/oropharyngeal tract; less than 5 microns to reach the lungs; and less than 3 microns to reach the deep lungs for local treatment or access via absorption to the systemic circulation. Further, particles that have impurities, surface imperfections and surface charges have a reduced tendency to agglomerate when formulated into dry powder, aqueous or nonaqueous formulations. Agglomeration increases particle size which prevents consistent or efficient delivery to the respiratory tract. Particles which have high purity, low surface energy, low surface imperfections and uniform size can be readily deaggregated when dispersed or suspended in fluid media. Such particles flow more easily or disperse more readily in fluid media including gases, vapors and liquids. By using nanomedicament dispersions or suspensions it is possible to achieve the fluid properties that contribute to optimum delivery.

In accordance with this invention, medicaments are fabricated into particles with narrow particle size distribution (usually less than 200 nanometers spread) with a mean particle hydrodynamic radius in the range of 50 nanometers to 700 nanometers. The nanomedicaments are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., “Phase Behavioral Effects on Particle Formation Processes Using Supercritical Fluids”, Pharmaceutical Research, vol. 16. p. 976 (1999). These methods permit the formation of micron and sub-micron sized particles with differing morphologies depending on the method and parameters selected. In addition, these nanoparticles can be fabricated by spray drying,, lyophilization, volume exclusion, and any other conventional methods of particle reduction.

Furthermore, these processes for producing nanometer sized particles, including SCF, can permit selection of a desired morphology (e.g., amorphous, crystalline, resolved racemic) by appropriate adjustment of the conditions for particle formation during precipitation or condensation. As a consequence of selection of the desired particle form, extended release of the selected medicament can be achieved. Also, fabricating the medicament into microspheres by volume exclusion induced precipitation can result in extended release profiles of the medicament to achieve specific pharmacokinetics and pharmacodynamic effects.

These particle fabrication processes are used to obtain nanoparticulates that have high purity, low surface imperfections, low surface charges and low sedimentation rates. Such particle features inhibit particle cohesion, agglomeration and also prevent settling in liquid dispersions. Additionally, because processes such as SF can separate isomers of certain medicaments, such separation would contribute to the medicament's enhanced activity, effectiveness as well as extreme dose reduction. In some instances, isomer separation also contributes to reduced side effects.

In accordance with the present invention, a compound is fabricated into a powdered form by any process including SCF, spray drying, precipitation and volume exclusion, directly into a collection media, wherein the particulate compound is thus automatically generated into a dispered or suspended formulation. This formulation may, in many instances, be the final formulation.

The invention provides nanoparticulates liquid dispersion and suspension formulations that can be delivered using jet, pneumatic and ultrasonic nebulisers, metered dose inhalers, dry powder inhalers, as well as other conventional pharmaceutical delivery systems.

As an example of formulation which can be made, a nanoparticulate liquid dispersion formulation comprised of (i) a saline solution; (ii) a preservative including chlorobutanol and benzylkonium chloride; and (iii) a suspending agent, including citrates and succinates, is within the scope of the invention. The invention further provides a nanoparticulate lyophilized particle that can be formulated using propellants such as 1,1,1,2,3,3,-heptafluoro-n-propane and/or 1,1,1,2-tetrafluoroethane or any mixture of both in any proportions, a surfactant and/or surface coating agent, and a trace amount of adjuvant. Such formulations can be delivered to the lung using a metered dose inhaler.

The adjuvant in the present invention is used to facilitate surfactant handling, while the surfactant in the present formulation invention is used to lubricate the valve in the formulation container and to facilitate the dispensability of medicament in the propellant.

Specific examples of formulations made in accordance with this invention, which contain the pharmaceutical agent budesonide and are intended for delivery directly to the lung, are set out below in Table 1 and are exemplary of the present invention:

TABLE I
Nanobudesonide Formulation Compositions Formulated by
SuperCritical Fluid Techniques

| Ingredient | Component | Range | Amount | Units |
|---|---|---|---|---|
| Formulation Number: 1 | | | | |
| Budesonide, EP | Active | 7.9-15.2 | 10.7 | mg |
| Tyloxapol, USP | Dispersant | 0.79-3.79 | 1.2 | mg |
| Benzalkonium chloride, | Preservative | 0.1-0.5 | 0.1 | mg |
| USP | | | | |
| Citric acid/Sodium Citrate | Buffer | | 2 | mM |
| WFI | | | q.s ad 1.0 | g |
| Formulation Number: 2 | | | | |
| Budesonide, EP | Active | 7.9-15.2 | 10.7 | mg |
| Isopropyl Myristate, USP | Dispersant | 0.79-3.79 | 1.2 | mg |
| Benzalkonium chloride, | Preservative | 0.1-0.5 | 0.1 | mg |
| USP | | | | |
| Citric acid/Sodium Citrate | Buffer | | 2 | mM |
| WFI | | | q.s ad 1.0 | g |
| Formulation Number: 3 | | | | |
| Budesonide, EP | Active | 7.9-15.2 | 10.7 | mg |
| Oleic Acid, USP | Dispersant | 0.79-3.79 | 1.2 | mg |
| Benzalkonium chloride, | Preservative | 0.1-0.5 | 0.1 | mg |
| USP | | | | |
| Citric acid/Sodium Citrate | Buffer | | 2 | mM |
| WFI | | | q.s ad 1.0 | g |
| Formulation Number: 4 | | | | |
| Budesonide, EP | Active | 7.9-15.2 | 10.7 | mg |
| Lecitin, USP | Dispersant | 0.79-3.79 | 1.2 | mg |
| Benzalkonium chloride, | Preservative | 0.1-0.5 | 0.1 | mg |
| USP | | | | |
| Citric acid/Sodium Citrate | Buffer | | 2 | mM |
| WFI | | | q.s ad 1.0 | g |
| Formulation Number: 5 | | | | |
| Budesonide, EP | Active | 7.9-15.2 | 10.7 | mg |
| Benzalkonium chloride, | Preservative | 0.1-0.5 | 0.1 | mg |
| USP | | | | |
| Citric acid/Sodium Citrate | Buffer | | 2 | mM |
| WFI | | | q.s ad 1.0 | g |
| Formulation Number: 6 | | | | |
| Budesonide, EP | Active | 7.9-15.2 | 10.7 | mg |
| Citric acid/Sodium Citrate | Buffer | | 2 | mM |
| WFI | | | q.s ad 1.0 | g |
| Formulation Number: 7 | | | | |
| Budesonide, EP | Active | 7.9-15.2 | 10.7 | mg |
| Chlorobutanol, USP | Preservative | 1.0-8.0 | 2.5 | mg |
| Citric acid/Sodium Citrate | Buffer | | 2 | mM |
| WFI | | | q.s ad 1.0 | g |
| Formulation Number: 8 | | | | |
| Budesonide, EP | Active | 7.9-15.2 | 10.7 | mg |
| Isopropyl Myristate, USP | Dispersant | 0.79-3.79 | 1.2 | mg |
| Chlorobutanol, USP | Preservative | 1.0-8.0 | 2.5 | mg |
| Citric acid/Sodium Citrate | Buffer | | 2 | mM |
| WFI | | q.s ad 1.0 | g | |
| Formulation Number: 9 | | | | |
| Budesonide | Active | 7.9-15.2 | 10.7 | mg |
| Polysorbate 80 | Dispersant | 0.79-3.79 | 1.2 | mg |
| Benzalkonium chloride | Preservative | 0.1-0.5 | 0.1 | mg |
| USP | | | | |
| Citric acid/Sodium Citrate | Buffer | | 2 | mM |
| WFI | | q.s ad 1.0 | g | |

Further examples are the production of dihydroergotamine or formoterol directly into a hydrofluorcarbon propellant system using supercritical process to make a metered dose inhaler formulation for the treatment of migraine or asthma.

| Ingredient | Component | Range | Amount | Units |
|---|---|---|---|---|
| Formulation Number: 10 | | | | |
| Dihydroergotamine | Active | 0.05-1.00 | 0.500 | mg |
| Isopropyl Myristate, USP | Dispersant | 0.000-0.100 | 0.005 | mg |
| HFA 227 | Propellant/ | 0.050-0.200 | 0.200 | mg |
| | suspending or | | | |
| | dispersing | | | |
| | media | | | |
| Formulation Number: 11 | | | | |
| Formoterol | Active | 0.05-0.050 | 0.005 | mg |
| Isopropyl Myristate, USP | Dispersant | 0.000-0.005 | 0.000 | mg |
| HFA 227 | Propellant/ | 0.050-0.200 | 0.100 | mg |
| | suspending or | | | |
| | dispersing | | | |
| | media | | | |

A recombinant human insulin can be produced directly into a hydrofluorcarbon propellant system using supercritical process to make a metered dose inhaler formulation for the treatment of diabetes. Altenatively insulin particles can be produced by volume exclusion precipitation directly into an aqueous phase carrier for to make a nebulizer inhaler formulation

| Ingredient | Component | Range | Amount | Units |
|---|---|---|---|---|
| Formulation Number: 12 | | | | |
| Insulin | Active | 0.05-2.00 | 1.00 | mg |
| Isopropyl Myristate, USP | Dispersant | 0.000-0.100 | 0.000 | mg |
| HFA 227 or 134a or | Propellant/ | 0.050-0.200 | 0.200 | mg |
| blends thereof | suspending | | | |
| | or dispersing | | | |
| | media | | | |
| Formulation Number: 13 | | | | |
| Insulin | Active | 0.05-2.00 | 1.00 | mg |
| Citric acid/Sodium | Buffer | | 0.1 | mg |
| Citrate | | | | |
| WFI | | | 2 | mM |
| | | | q.s ad 1.0 | g |

For the metered dose inhalers the aerosol formulation can be manufactured in accordance with the present invention by first preparing a kettle with a liquid propellant, surfactant and adjuvant. The nanomedicament is then collected directly into the kettle by lyophilization of the nanoparticulates. These materials are then mixed. The resulting dispersion is then added to a canister, crimpled with a valve, by forcing the dispersion through the valve by pressure filling. The canister containing the aerosol formulation is then sonicated to assure thorough mixing and surfactant-medicament surface wetting. This invention applies to any form of scale-ups employing cold and pressure filling.

By use of the present invention, significant efficiencies in time and expense are achieved. Since the active compound is produced in particulate form directly into a fluid comprising all or part of the final carrier vehicles, it is not necessary to first store and then later re-suspend the formed particles. Moreover, once the nanoparticles are allowed to precipitate, they tend to agglomerate. Suspending such agglomerated particles presents many difficulties due to the need to overcome the cohesive forces between the molecules. Such formulating difficulties are overcome in the present invention since the particulate compound is directly formed into the carrier, thus avoiding the need to re-suspend the particles.

What we claim is:

1. A method for preparing a formulation containing nanoparticles of a compound comprising:
forming the compound into nanoparticles; and
delivering said nanoparticles as they are generated directly to a collection media, wherein said collection media is a suspending or dispersion media and a desired component of the formulation.

2. The method according to claim 1, wherein the formulation is a pharmaceutical formulation.

3. The method according to claim 1, wherein the formulation is a pharmaceutical formulation for to the respiratory tract via inhalation

4. The method according to claim 2 wherein the compound is a medicament.

5. The method according to claim 4, wherein the medicament is selected from the group consisting of anti-allergic, anti-inflammatory, steroid, anti-cholinergic, mucolytic, and/or beta-agonist agents, or combinations thereof.

6. The method according to claim 4, wherein the medicament is selected from the group consisting of salbutamol, salmeterol, formeterol, fenterol, fluticasone dipropionate, beclomethasone dipropionate, dexamethasone, budesonide, flunisolide, ciclesonide, triamcinolone, sodium cromolyn, ipratropium and their salts or solvates.

7. The method according to claim 4 wherein the medicament is selected from the group consisting of an anti-cancer, anti-emetic, anti-migraine, narcotic analgesic, antipsychotic, anti-depressant, analgesic, anti-inflammatory, antineoplastic, antibiotic, anti-infective, or antidiuretic agents.

8. The method according to claim 4 wherein the medicament is budesonide. (also claims specifically for dihydroergotamine, formoterol, and insulin)

9. The method of claim 1 further comprising aerosolization of the formulation.

10. The method of claim 1, wherein the nanoparticles are formed using a method selected from spray-drying and supercritical fluid, precipitation or volume-exclusion precipitation.

11. The method according to claim 10, wherein the supercritical fluid method is RESS or SEDS.

12. The method of claim 1 further comprising separating isomers of the compound.

13. The method of claim 2, wherein the formulation is stored in a canister for subsequent local delivery to a patient.

14. The method of claim 1 wherein the compound is not water soluble, or has low solubility in water.

15. The method of claim 1 wherein the compound is water soluble.
